(19) United States
(12) Patent Application Publication
(10) Pub. No.: US 2022/0000474 A1
(43) Pub. Date: Jan. 6, 2022

(54) APPARATUS FOR TRANS-OSSEOUS ROTATOR CUFF REPAIR

(71) Applicant: Banarji B H, Bangalore (IN)

(72) Inventor: Banarji B H, Bangalore (IN)

(73) Assignee: Banarji B H, Bangalore (IN)

(21) Appl. No.: 17/013,558

(22) Filed: Sep. 5, 2020

(30) Foreign Application Priority Data
Jul. 4, 2020 (IN) 202041028489

(51) Int. Cl. A61B 17/04; A61B 17/06

(52) U.S. Cl. CPC A61B 17/0482; A61B 2017/06071; A61B 17/06066

(57) ABSTRACT

A kit for trans-osseous rotator cuff repair comprising, a handle (101) with a hollow guide (106) and a first connector (104A), a hollow sleeve (103) that passes through the hollow guide (106) in the handle (101), a suture guider (105 and 503) to guide a surgical suture (516) through the hollow sleeve (103), an adaptable hook (107A and 107B) with a narrow slit (118) at one end and a second connector (104B) at opposite end to couple with the first connector (104A), and a pin (901) with first and second apertures (906 and 908) enabling knotless suture of a soft tendon tissue to a bone, wherein the hollow sleeve is adjustable within the hollow guide (106) and a tunnel is created within the bone by creating perpendicularly intersecting a first and a second hole (718 and 706) in that the narrow slit (118) of the adaptable hook (107A and 107B) retrieves the surgical suture (516) from the second hole (706) through the first hole (718) of the tunnel.

### BACKGROUND

### Cross References to Related Applications

This application claims priority from Indian patent application No. 202041028489 filed on Jul. 4, 2020 which is incorporated herein in its entirety by reference

### TECHNICAL FIELD

Embodiments of the present disclosure relate generally to a medical device and more specifically to an apparatus for trans-osseous rotator cuff repair.

### RELATED ART

Rotator cuff is a group of muscles and tendons that surrounded the shoulder joint and keeps the upper arm bone firmly in place within a shallow socket of the shoulder. A rotator cuff injury causes ache in the shoulder and gets worsened with further use of the arm away from the body. These injuries are quite common in people who are extensively performing overhead activities besides the age factor. In certain circumstances, rotator cuff tears may occur which requires an immediate medical evaluation to determine if a surgery is required to treat the injury as progressive degeneration of tendons tissues leads to extensive damage of the tendons.

Various mechanisms, devices and apparatus for rotator cuff repair are available in the prior art. However, all the apparatus and implemented mechanisms in the prior art are extensive, complicated and highly expensive.

Traditionally, rotator cuff repair surgery is performed as an open procedure where a tunnel is made into a bone and sutures are passed across the tunnel and through the torn tendon before tying the knots. This is an extensive open surgery.

With advent of technology and introduction of arthroscopy, an arthroscopic rotator cuff repair evolved with use of suture implants loaded with sutures. These implants are placed in the bone tunnel that are made with a greater traversing area. As conventional knot tying results in bulky knots and is time consuming, knotless suture implants are introduced. However, conventional knotless suture implants are inefficient as they get loosen and pulled out over a period of time or during the surgery leading to suture cut through the tendon.

Thus, few researchers developed an arthroscopic trans osseous rotator cuff repair technique in which the bone tunnel is made and sutures are passed across the tunnel and through the rotator cuff tendon where the knots are tied automatically. However, conventional arthroscopic trans osseous rotator cuff repair mechanism is limited to use as they are expensive and the strong sutures may cut through the osteoporotic bone causing further damage to the rotator cuff injury.

Hence, there is a need for an efficient, stable and reliable apparatus to perform the rotator cuff repair without causing any damage to the bone.

### SUMMARY

According to an aspect of the present disclosure, a kit for trans-osseous rotator cuff repair comprising, a handle (101) with a hollow guide (106) and a first connector (104A), a hollow sleeve (103) that passes through the hollow guide (106) in the handle (101), a suture guider (105 and 503) to guide a surgical suture (516) through the hollow sleeve (103), an adaptable hook (107A and 107B) with a narrow slit (118) at one end and a second connector (104B) at opposite end to couple with the first connector (104A), and a pin (901) with first and second apertures (906 and 908) enabling knotless suture of a soft tendon tissue to a bone, wherein the hollow sleeve is adjustable within the hollow guide (106) and a tunnel is created within the bone by creating perpendicularly intersecting a first and a second hole (718 and 706) in that the narrow slit (118) of the adaptable hook (107A and 107B) retrieves the surgical suture (516) from the second hole (706) through the first hole (718) of the tunnel.

Several aspects are described below, with reference to diagrams. It should be understood that numerous specific details, relationships, and methods are set forth to provide a full understanding of the present disclosure. One who skilled in the relevant art, however, will readily recognize that the present disclosure can be practiced without one or more of the specific details, or with other methods, etc. In other instances, well-known structures or operations are not shown in detail to avoid obscuring the features of the present disclosure.

### BRIEF DESCRIPTION OF DRAWINGS

FIG. 1A through 1E are the diagrams illustrating various tools that are employed either individually or in combination for trans-osseous rotator cuff repair in an embodiment of the present disclosure.

FIG. 2 is a diagram illustrating plurality of tips in adaptable hook of the jig in an exemplary embodiment.

FIGS. 3A and 3B are the diagrams illustrating an assembly of the adaptable hook to the handle of the jig in another embodiment of the present disclosure.

FIGS. 4A and 4B are the diagrams illustrating the assembly of hollow sleeve into the handle of the jig in yet another embodiment of the present disclosure.

FIG. 5A is a diagram illustrating a supplement suture guider through the sleeve of the jig in another embodiment of the present disclosure.

FIG. 5B through 5D are the diagrams illustrating the mechanism involved in releasing the suture after guiding it through the tip of the adaptable hook in another embodiment of the present disclosure.

FIGS. 5E and 5F are the diagrams illustrating a suture guider for guiding the suture into the adaptable hook of the jig in yet another embodiment of the present disclosure.

FIGS. 6A and 6B are the diagrams illustrating the passage of suture through the tip of the adaptable hook in an embodiment of the present disclosure.

FIG. 7 is a schematic diagram illustrating the creation of tunnel in the bone with perpendicular intersecting holes using jig of the present disclosure.

FIG. 8 is a diagram illustrating the retrieval of suture through the tunnel using adaptable hook of the present disclosure.

FIG. 9 is a diagram illustrating a pin enabling the implementation of knotless sutures in yet another embodiment of the present disclosure.

FIG. 10 is a diagram illustrating the steps involved in implementing knotless sutures in rotator cuff repair using the pin of the present disclosure.

### DETAILED DESCRIPTION OF THE PREFERRED EXAMPLES

A kit comprising a plurality of surgical tools (also referred to as apparatus) for trans-osseous rotator cuff repair is provided in the present disclosure. The kit enables the surgeon to drill the tunnel in bone with perpendicular intersecting holes and retrieve the sutures through tunnel effortlessly. Also, a pin provided within the kit enables the knotless sutures for trans-osseous rotator cuff repair i.e., re-attaching the tendon soft tissue to the bone without causing any damage to the tissue and the bone.

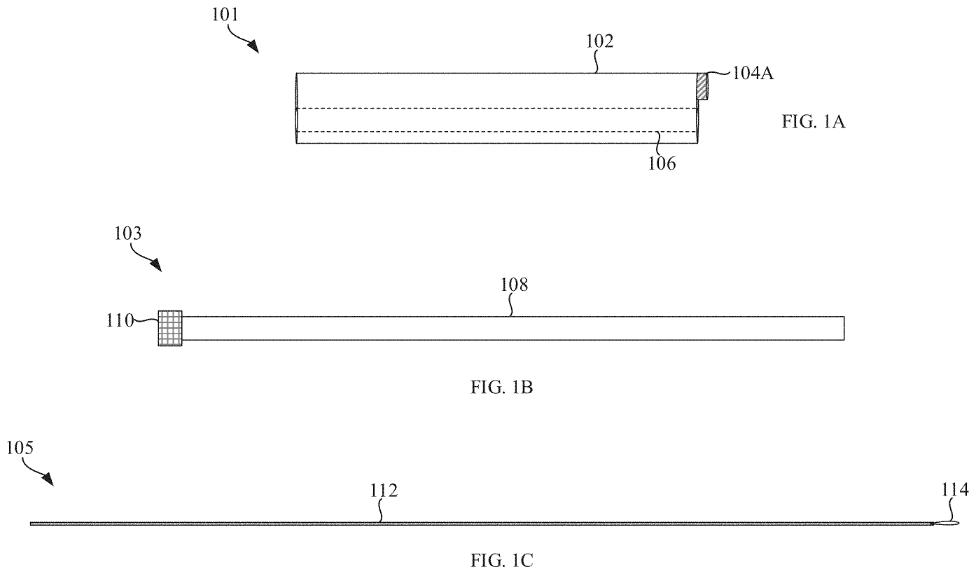

FIG. 1A through 1E are the diagrams illustrating various tools that are employed either individually or in combination for trans-osseous rotator cuff repair in an embodiment of the present disclosure. FIG. 1A illustrates a handle (101) used for forming a jig in an embodiment of the present disclosure. The handle (101) comprises a solid metallic tube provided with a hollow guide (106) and a first connector (104A). In an embodiment, the hollow guide (106) guides a conventional drill bit, surgical screw driver and other surgical instruments to pass through and reach a desired destination. The first connector (104A) is configured to connect to an external equipment to enable hassle-free handling of the jig after assembly of all the tools provided in the kit of the present disclosure. In an embodiment, the first connector (104A) comprises at least one of an adjustable screw, bolt, nut and the like for levelling the external equipment or other tools. In an example, the length and width of the handle (101) may vary with respect to dimensions of the bone as well as to comply with prerequisites of trans-osseous rotator cuff repair.

FIG. 1B is a diagram illustrating a hollow sleeve (103) that passes through the hollow guide (106) in the handle (101). The hollow sleeve (103) comprises a hollow tube (108) coupled with a knob (110) that helps in adjusting the movement of the hollow sleeve (103) within the hollow guide (106). In an embodiment, the knob (110) is configured to adjust and fasten the hollow sleeve (103) in a firm position as desired. In another embodiment, the hollow sleeve (103) is having dimension of at least 1 mm less than that of the dimensions of the hollow guide (106). In an example, the length of the hollow sleeve (103) may be extended by coupling an additional hollow sleeve either at the front or rear of the hollow sleeve (103).

FIG. 1C is a diagram illustrating a suture guider (105) to guide a suture through the hollow sleeve (103) within the handle (101). The suture guider (105) comprises a thin metal bar (112) with a loop (114) at the tip which holds the suture and carries to the extent it passes through the hollow sleeve within the handle (101). The suture guider (105) may also comprise a conventional suture holder that carries a double layered loop of suture through the hollow sleeve (103).

FIGS. 1D and 1E are the diagrams illustrating adaptable hooks (107A and 107B) in an embodiment of the present disclosure. The adaptable hooks (107A and 107B) are configured to guide the drill bit to create a first hole intersecting a second hole in which tip of the hook (107A and 107B) is placed. In an embodiment, the first and second holes are perpendicular to each other and intersects at a point forming a tunnel within a bone. Further, the adaptable hooks (107A and 107B) are configured to retrieve a suture entered from the first hole through the second hole. The adaptable hook (107A) comprises a curved metallic rod (116) with a narrow slit (118) at one end and a second connector (104B) at the opposite end. In an embodiment, the second connector (104B) is configured and compatible to couple with the first connector (104A) while the curved metallic rod (116) may be changed to a multi-step (120 as shown in FIG. 1E) or any desired shape that fits the shoulder arm of a person. The shape of the curved metallic rod (116) may be selected by user of the tool and the second connector (104B) is fastened to the selected metallic rod (116) by conventional mechanical means. The narrow slit (118) enables the sutures to get stuck into it while retrieving it from the tunnel created in the bone.

In another embodiment, length and angle of the adaptable hook (107A and 107B) may be adjusted as desired by using the first and second connectors (104A and 104B) in such a way that the narrow slit (118) is in line with a skeletal (or central) axis of the hollow sleeve (103).

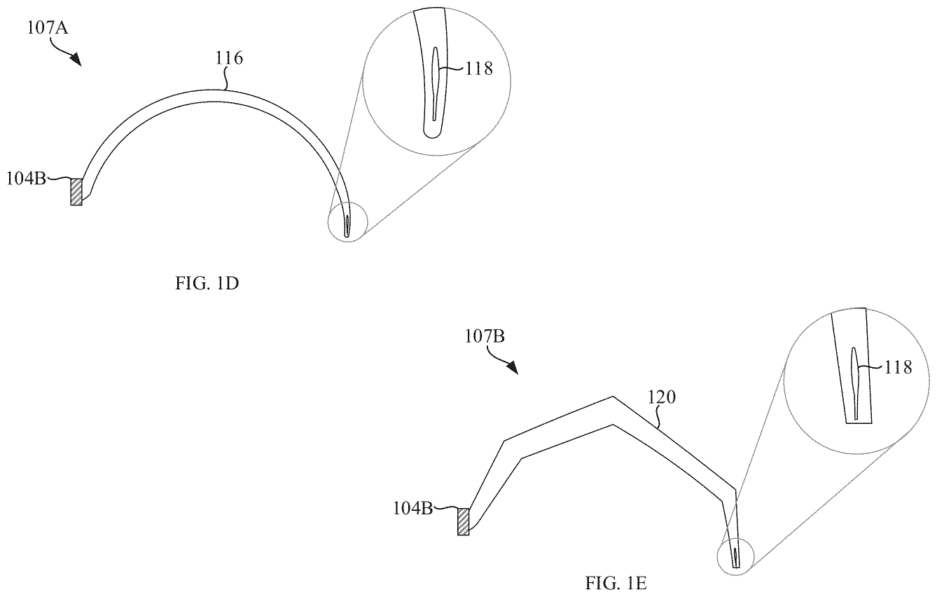

FIG. 2 is a diagram illustrating plurality of tips in adaptable hook of the jig in an exemplary embodiment. As discussed in the previous embodiment, the adaptable hook (107A and 107B) comprises a narrow slit at one end of the metallic rod (116). In an example, the narrow slit may comprise of plurality of shapes in which the slit becomes narrower from top to bottom. As shown there, 201 through 209 represents plurality of shapes wherein all the shapes are broader at the top while it becomes narrow at the bottom. The broader section of the slit (118) helps in passage of sutures through it while the narrow section of the slit at the bottom hold the sutures firmly in place without letting it slip away through the broader section.

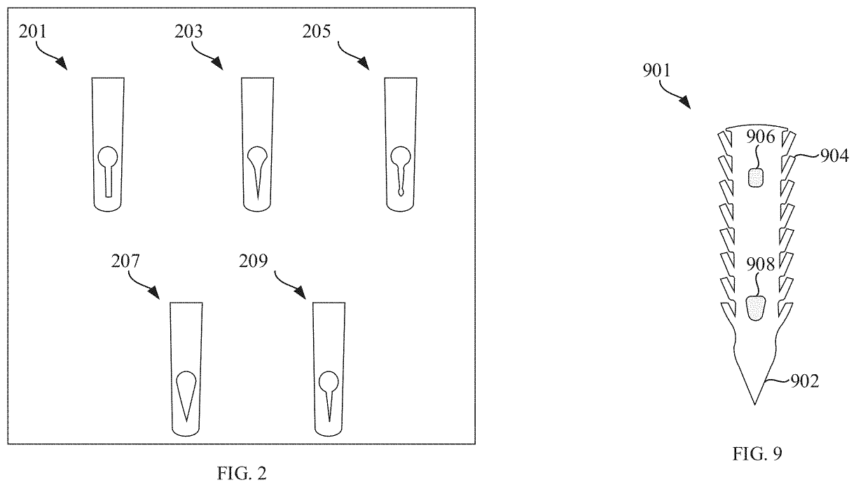

FIGS. 3A and 3B are the diagrams illustrating an assembly of the adaptable hook to the handle (302) of the jig (301 and 303) in another embodiment of the present disclosure. As shown there, the adaptable hooks (306A and 306B) are coupled to the handle (302) of the jig provided in the kit of the present disclosure by using the first and second connectors. The first and the second connectors are coupled together and forms a connecting means (308) which allows the adaptable hooks to be placed in a specific direction aligned with the handle (302). In an embodiment, the connecting means (308) helps in fastening or positioning the narrow slit (312) of the adaptable hooks (306A and 306B) in line with skeletal axis (310) of the hollow guide (304). As shown there, 301 represents a jig with curved hook (306A) while 303 represents a jig with multi-step hook (306B) wherein narrow slit (312) of both the hooks are in line with skeletal axis (310) of the hollow guide (304) in the handle (302).

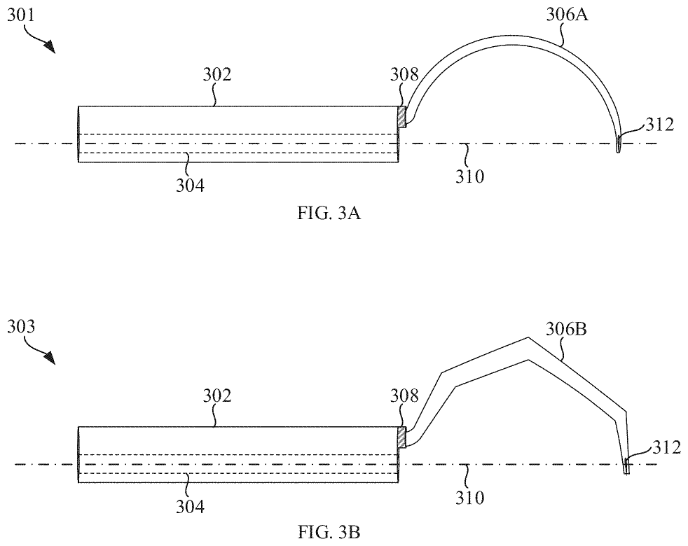

FIGS. 4A and 4B are the diagrams illustrating the assembly of hollow sleeve into the handle of the jig in yet another embodiment of the present disclosure. As shown in 401, the jig comprises a handle (402), an adaptable curved hook (406) and a hollow sleeve (412) passing through the hollow guide (410) of the handle (402) assembled together in a similar way to that of the assembly of tools as discussed in the previous embodiments. The connecting means (404) holes the adaptable curved hook (406) in a firm position with the narrow slit (408) in line with the skeletal axis of the hollow guide (410). In an embodiment, the knob (414) of the hollow sleeve (412) allows the movement of the sleeve (412) to- and-fro within the hollow guide (410) such that the movement is restricted to a predefined length (416) for example, say the sleeve (412) is movable up to the surface of the narrow slit of the adaptable hook (406). In another embodiment, the predefined length (416) may be adjusted by fastening the knob (414) over the sleeve (412).

As shown in 403, the adaptable hook (406) is placed into a drilled hole within a bone (420) in such a way that the tip i.e., narrow slit (408) goes inside the vertical hole in the bone (420). After fixing the adaptable hook (406), the adjustable hollow sleeve (412) is adjusted to move up to the surface of the bone (420) by applying either an external force (418) or fastening the knob (414). This allows the user to pass through a drill bit for making the first hole that intersects with the second hole where the narrow slit is present. Thus, the intersection point of the first and second hole is achieved effortlessly by adjusting the sleeve (412) within the handle (402).

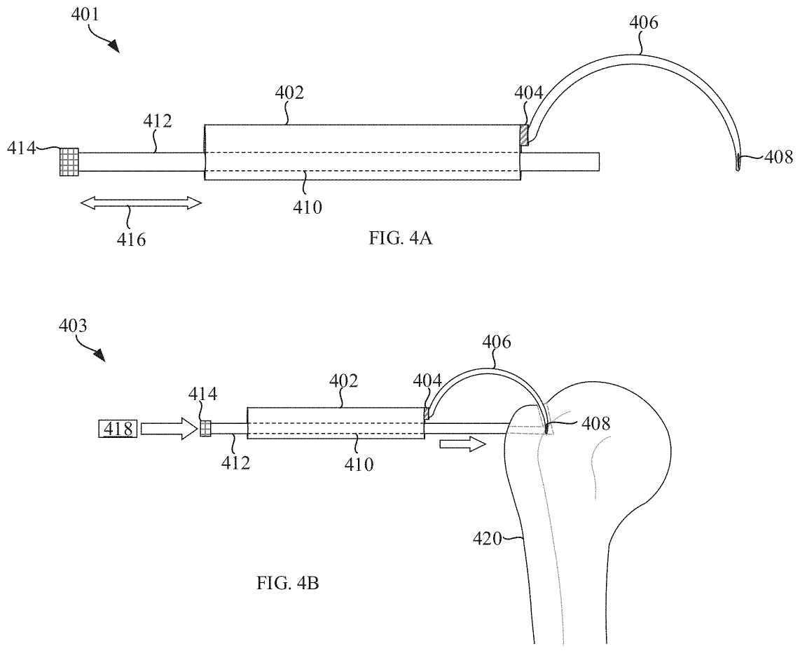

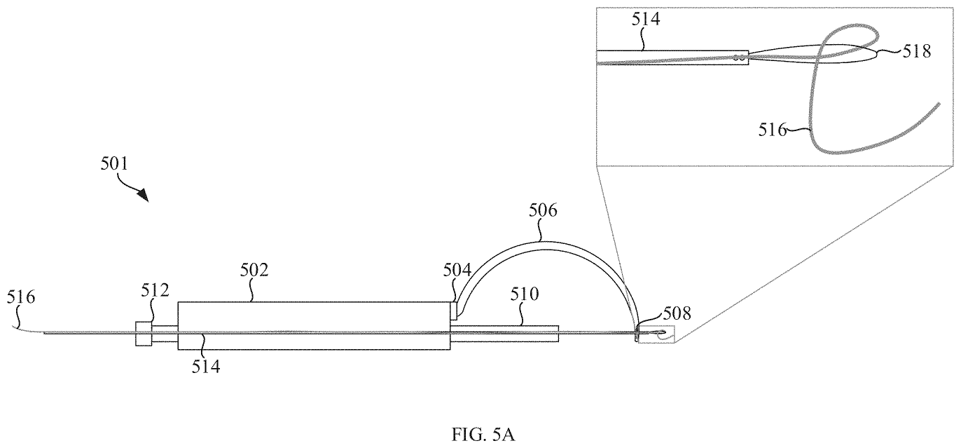

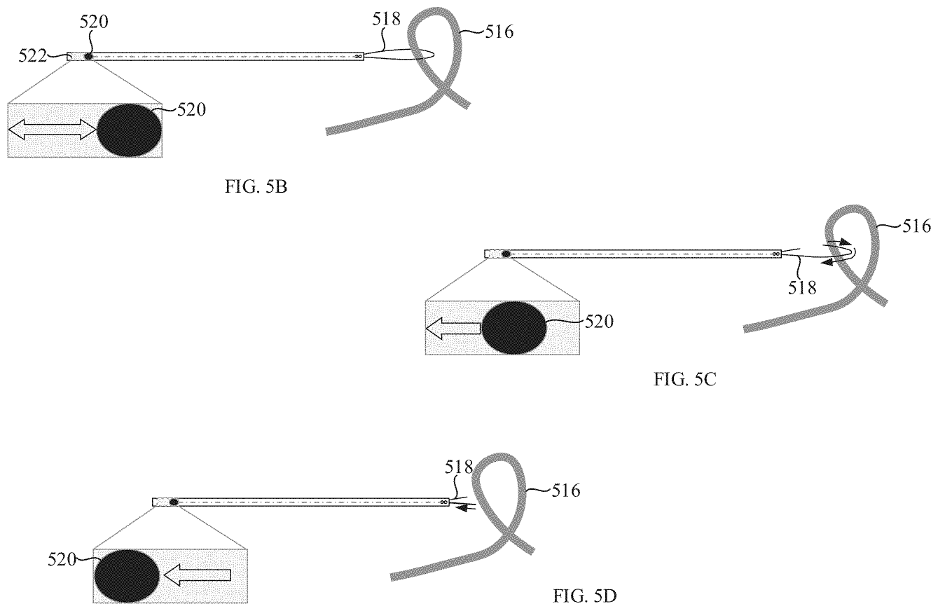

FIG. 5A is a diagram illustrating a supplement suture guider through the sleeve of the jig in another embodiment of the present disclosure. As shown in 501, the jig assembly comprises the handle (502), the adaptable hook (506), connecting means (504) that connects the adaptable hook and the handle, the hollow sleeve (510) and a supplement suture guider (514) loaded with a surgical suture (516). The tools in the assembly 501 are operable and functional in a similar way to that of the tools as discussed in the FIG. 1A through 1E. As shown there, the supplement suture guider (514) comprises a loop (518) at the tip which is configured to hold the suture (516) and carry it through the hollow sleeve (510) into the narrow slit (508) of the adaptable hook (506). In an example, the suture (516) may be introduced into the narrow slit (508) through the hollow sleeve (510) as a plurality of layers for example, double layered, triple layered sutures and the like. The supplement suture guider (514) is pushed manually towards the narrow slit of the adaptable hook (506) such that the loop (518) pass through the broader section of the narrow slit (508) and releases the suture there before coming out of the slit (508). The mechanism involved in releasing the suture by supplement suture guider is further described in the following figures.

FIG. 5B through 5D are the diagrams illustrating the mechanism involved in releasing the suture after guiding it through the tip of the adaptable hook in another embodiment of the present disclosure. In an embodiment, the supplement suture guider (514) further comprises an auto retracting button (520) that is configured to retract the loop (518) releasing the contents that are held within the loop. As shown there, a slot (522) with the retracting button (520) is provided on surface of the suture guider (514) towards the knob (512) of the hollow sleeve (510). As shown in the FIG. 5B, the retracting button (520) is in its initial position such that the loop (518) remains closed and hold the suture (516) in it. As shown in the FIG. 5C, the retracting button is getting pulled towards the knob (512) i.e., away from the loop (518). This results in opening of the loop (518) wherein the suture (516) is about to get released. As shown in the FIG. 5D, the retracting button is completely pulled away from the loop (518) within the slot (522) which completely retracts the loop by releasing the suture (516). As soon as the suture (516) gets released, it falls into the narrow section of the slit (508) from the broader section. Further, the retracting button automatically reaches its initial position by closing the loop (518) with the help of a compression spring. Thus, the suture (516) from the supplement suture guider (514) gets released and stuck within the narrow slit (508) of the adaptable hook (506).

FIGS. 5E and 5F are the diagrams illustrating a suture guider (503) for guiding the suture into the adaptable hook of the jig in yet another embodiment of the present disclosure. The suture guider (503) in yet another embodiment of the present disclosure comprises a handle (532), a retracting knob or button (534), a passage (536), a connecting bar (538) and a shaft (540). In an embodiment, the suture guider (103 and/or 503) is able to be sterilised and is made of at least one of a known surgically accepted material.

The handle (532) is configured to drive the shaft (540) in a specific direction as desired and the handle may comprise at least one of metallic and non-metallic material that is able to be sterilised after using it. The connecting bar (538) couples the handle (532) and the shaft (540) together for performing a desired operation. The shaft (540) comprises a ‘u’ shaped groove (542) at an extreme tip away from the handle (532). The groove (542) holds the surgical suture firmly and pulls along with it in a direction of a force applied onto the handle (532). The retracting knob (534) and the passage (536) are configured to secure and/or release a tension in a surgical suture.

As shown in the FIG. 5F, the surgical suture (544) is passed from the retracting knob (534) to the groove (542) of the shaft (540) and then returned to the passage (536) where it gets coupled to the knob (534) in such a way that the surgical suture (544) is retracted by operating the retracting knob (534). The retracting knob (534) is operated in such a way that the tension in the surgical suture (544) may be secured or released along the length of the shaft (542) passing through the grove (542) and the passage (536). In an embodiment, the suture guider (503) is introduced into the hollow sleeve of the present disclosure in such a way that the groove (542) is passed into the narrow slit of the adaptable hook. Releasing tension in the suture by operating the retracting knob (534) leads the suture (544) to get stuck within the narrow slit.

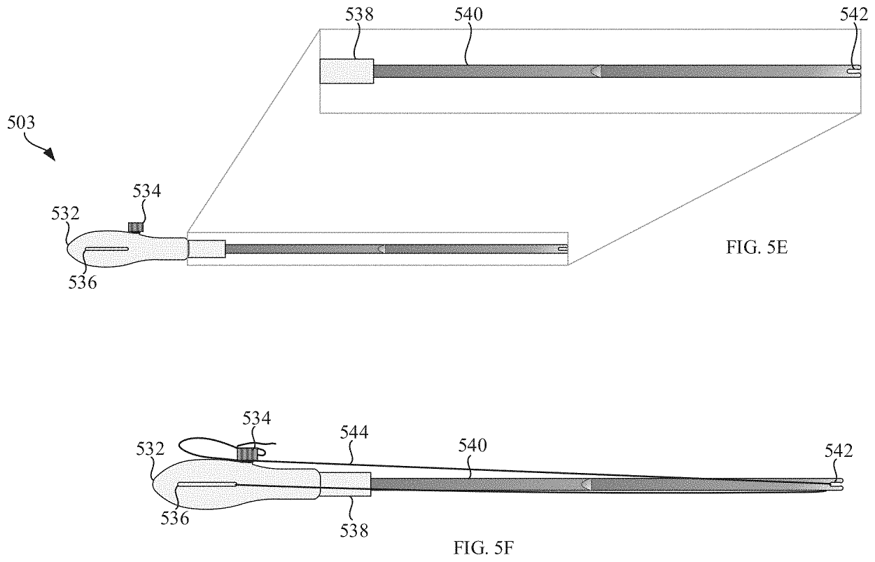

FIGS. 6A and 6B are the diagrams illustrating the passage of suture through the tip of the adaptable hook in an embodiment of the present disclosure. As shown in 601 and 603, the tools (as discussed in FIG. 1A through 1E) are assembled together forming a jig of the present disclosure. The tools comprise the handle (602), adaptable hook (606), connecting means (604) connecting the handle and the adaptable hook, hollow sleeve (610), knob (612) configured to adjust the position of the sleeve (610) and suture guider (614) loaded with the surgical suture (616). All these tools are functional and operable in a similar way to that of the tools as discussed in FIG. 5A through 5D. As shown in 601, the suture guider (614) carries the suture (616) through the narrow slit (608) of the adaptable hook (606). Once the suture is released from the loop of the suture guider (614) (as discussed in FIG. 5B through 5D), the suture guider (614) is pulled out of the slit (608) from the hollow sleeve (610). As shown in 603, the suture (616) gets dropped from broader section of the slit (608) into the narrow section that results in the suture gets stuck in the slit (608).

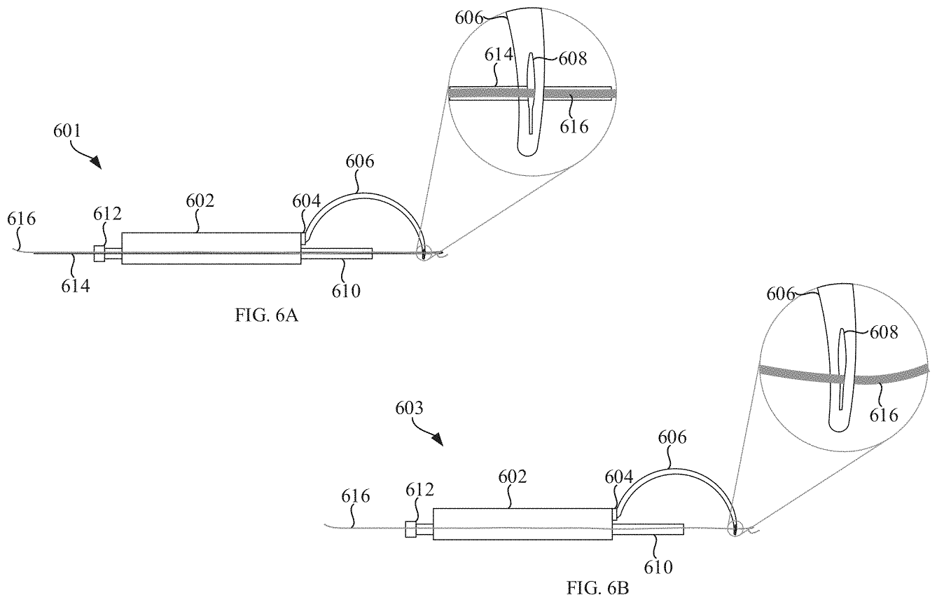

FIG. 7 is a schematic diagram illustrating the creation of tunnel in the bone with perpendicular intersecting holes using jig of the present disclosure. In order to repair rotator cuff injury, one has to drill two holes in the bone to pass through a suture from the holes to hold the soft tendon tissue. In present embodiment of the disclosure, a first hole (718) and a second hole (706) are created by using tools (as discussed in the FIG. 1A through 1E) of the present disclosure or conventional surgical drill bits. As shown in 701 and 703, the second hole (706) is drilled first by using a conventional surgical drilling unit (704) vertically from upper surface of the bone (702). In order to drill the first hole on lateral axis, the tools of the present disclosure or a conventional surgical drill bit is used so that the first and second holes (718 and 706) meet at an intersection point (720). In an example, dimensions of the first and second holes (718 and 706) may vary from each other and are created as per the requirement of the surgeon to perform the rotator cuff repair. As shown in 705, the tools are assembled to form a jig and the adaptable hook (710) is inserted into the second hole (706) by holding the handle (708). Then a surgical drill bit is inserted into the hollow sleeve (716) which is positioned to touch the lateral surface of the bone (702) by using the knob (714). The drill bit is then operated as per the prescribed parameters until it reaches the tip i.e., narrow slit of the adaptable hook (710). This facilitates an intersection (720) of the second and first holes (706 and 718) forming a tunnel within the bone without causing any loss or damage to the tissue. As shown in 707, 718 represents the first hole and 706 represents the second hole wherein both of them are intersected at 720.

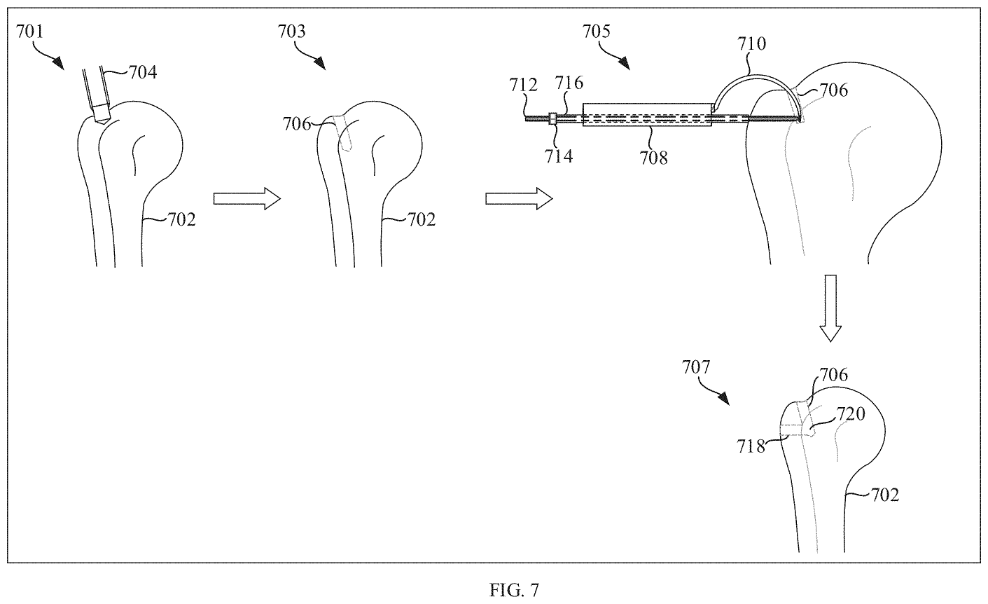

FIG. 8 is a diagram illustrating the retrieval of suture through the tunnel using adaptable hook of the present disclosure. As shown in 801, the assembled tools of the present disclosure are used to pass through a suture (814) into the slit (806) of the adaptable hook (804). Once, the first and second holes are drilled forming a tunnel as discussed in the FIG. 7, the adaptable hook is kept in the second hole (818) by holding the handle (802). The suture guider (812) carrying the suture (814) is then inserted into the hollow sleeve (808). The suture guider (812) is allowed to enter into the first hole (820) inside the bone until it passes through the narrow slit (806) of the adaptable hook (804).

As shown in 803, the retracting button of the suture guider (812) is operated to release the suture and set it free before taking the suture guider (812) outside the hollow sleeve (808). The hollow sleeve that is in contact with the surface of the bone is then released and pulled away from the bone (816) using knob (810). Then the handle (802) of the jig is lifted upwards in such a way that the hook gets lifted vertically from the second hole (818) wherein the suture is still passing through the first hole (820) from the hollow sleeve (808). This results in passing of suture from first hole (820) to the second hole (818) through the tunnel which has been retrieved by using the adaptable hook (804). This enables the surgeon to perform the knots and further procedural aspects easily. In an example, the retrieved sutures may be tied with knots or automatically fixed by using conventional suture implants within the tunnel of the bone. In another example, the sutures may be coupled to a conventional disc that is adhered to the opening of the tunnel which holds the sutures firmly.

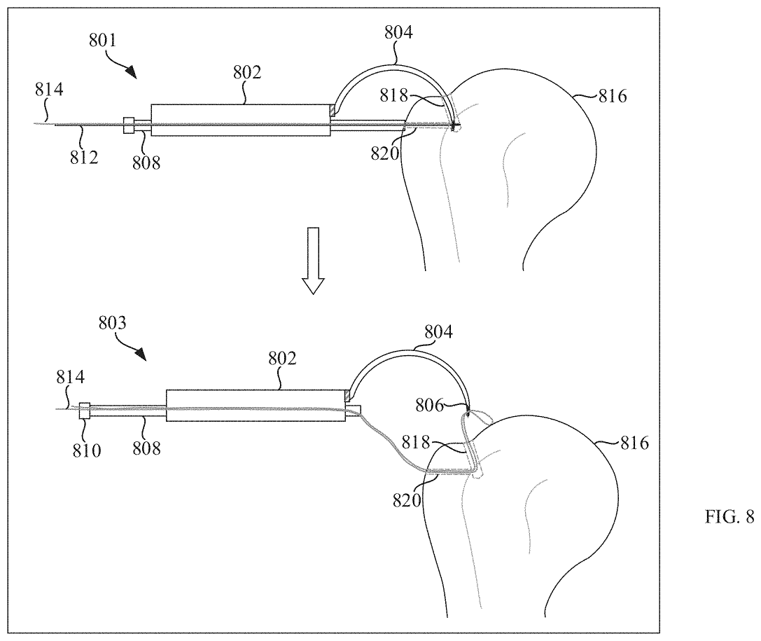

FIG. 9 is a diagram illustrating a pin enabling the implementation of knotless sutures in yet another embodiment of the present disclosure. As shown there the pin (901) comprises a pointed head (902) with plurality of spikes like structures (904) on the side walls. In an embodiment, the pointed head (902) helps in inserting the pin into a drilled hole within the bone easily while the spike structures (904) helps the pin (901) to get stuck to the walls of the bone within the drilled hole. The spike structures firmly hold the pin within the hole and allows the user to implement knotless sutures for rotator cuff repair. Further, the pin (901) comprises a pair of apertures (906 and 908) that are configured to increase the tension to fasten the sutures without tying knots. The mechanism involved in implementing knotless sutures using the pin (901) is further explained in the following figures.

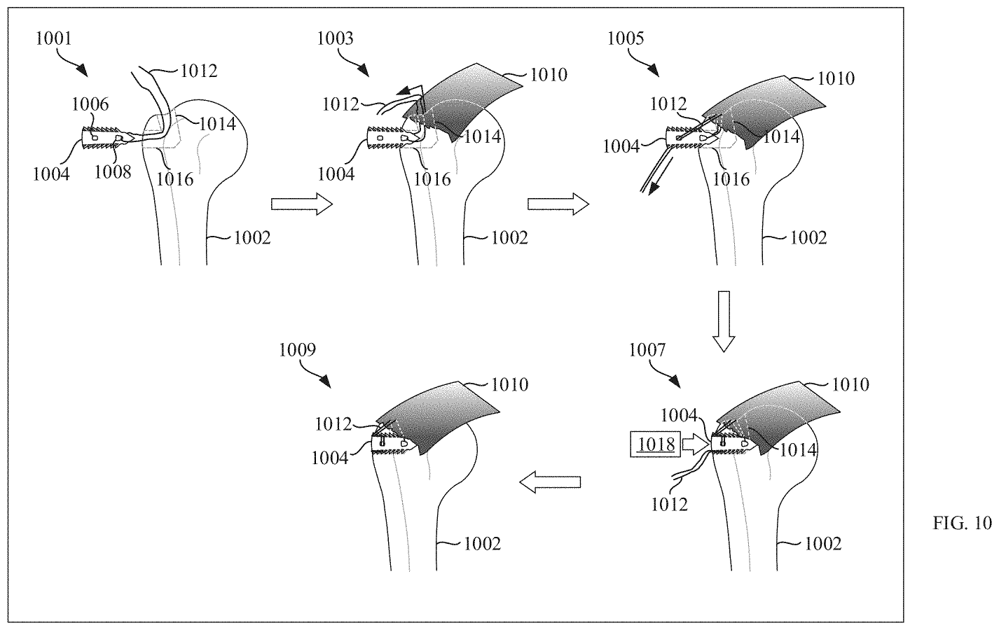

FIG. 10 is a diagram illustrating the steps involved in implementing knotless sutures in rotator cuff repair using the pin of the present disclosure. As shown in 1001, a first and second holes (1016 and 1014) are drilled within the bone (1002) using the tools of the present disclosure as discussed in the previous embodiments. In an embodiment, the suture (1012) is first passed through the second aperture (1008) i.e., bottom aperture close to the pointed head of the pin (1004) and the two ends are then pulled from the first hole (1016) to outer surface of the bone (1002) through the second hole (1014) using the adaptable hook of the present disclosure. In 1003, both the ends of the sutures that are coming out of the second hole (1014) are inserted into the first aperture (1006) of the pin (1004) by passing them through the soft tendon tissue (1010). This makes the pointed head of the pin (1004) to get inserted into the first hole (1016).

In 1005, both ends of the suture (1012) are pulled at the first aperture (1006) so that the suture passing through the tunnel inside the bone (1002) as well as the soft tendon tissue (1010) gets fastened without causing any damage to the tissue. In 1007, back of the pin (1004) is tapped gently by application of an external force (1018) so that the pin (1004) gets completely inserted into the first hole (1016) which leads to the fastening of the suture. As the suture gets fastened, it pulls the soft tendon tissue (1010) towards the bone wherein left-over ends of the suture comes outside from the first aperture of the pin. As the first aperture gets stuck inside the bone tissue within the first hole, the sutures also get stuck to the peripheral walls of the bone and gets fastened automatically. In 1009, the left-over ends that are coming out from the first aperture of the pin are removed completing the repair of rotator cuff injury without causing any damage to the other tissues.

While various embodiments of the present disclosure have been described above, it should be understood that they have been presented by way of example only, and not limitation. Thus, the breadth and scope of the present disclosure should not be limited by any of the above-discussed embodiments but should be defined only in accordance with the following claims and their equivalents.

What is claimed is:

1. A kit for trans-osseous rotator cuff repair comprising:
a handle (101) with a hollow guide (106) and a first connector (104A);
a hollow sleeve (103) that passes through the hollow guide (106) in the handle (101);
a suture guider (105 and 503) to guide a surgical suture (516) through the hollow sleeve (103);
an adaptable hook (107A and 107B) with a narrow slit (118) at one end and a second connector (104B) at opposite end to couple with the first connector (104A); and
a pin (901) with first and second apertures (906 and 908) enabling knotless suture of a soft tendon tissue to a bone,
wherein the hollow sleeve is adjustable within the hollow guide (106) and a tunnel is created within the bone by creating perpendicularly intersecting a first and a second hole (718 and 706) in that the narrow slit (118) of the adaptable hook (107A and 107B) retrieves the surgical suture (516) from the second hole (706) through the first hole (718) of the tunnel.

2. The kit as claimed in claim 1, wherein dimensions of the hollow sleeve (103) is 1 mm less than that of the hollow guide (106) to enable free movement of the hollow sleeve (103).

3. The kit as claimed in claim 2, wherein the suture guider (105) comprises a loop (518) and a retractable button (520) that are operable to hold as well as release the surgical suture (516) within the loop (518).

4. The kit as claimed in claim 3, wherein the suture guider (105 and 503) carries the surgical suture (516) from outside into the narrow slit (118) through the first hole (718) and releases it in the narrow slit (118) of the adaptable hook (107A and 107B).

5. The kit as claimed in claim 4, wherein the narrow slit (118) of the adaptable hook (107A and 107B) comprises a wider area at its top while a narrow area at its bottom in that the wider area is always in line with skeletal axis (310) of the hollow guide (106).

6. The kit as claimed in claim 5, wherein the surgical suture (516) released by the suture guider (105 and 503) gets stuck within the narrow area at bottom of the narrow slit (118) of the adaptable hook (107A and 107B).

7. The kit as claimed in claim 6, wherein the pin (901) comprises a pointed head (902) with plurality of spikes (904) on peripheral sides that are configured to hold the pin (901) firmly when inserted into the bone.

8. The kit as claimed in claim 7, wherein the surgical suture (516) passes through the second aperture (908) of the pin (901) and then to the first aperture (906) through a soft tendon tissue (1010) such that tapping the pin (901) into the tunnel of the bone fastens and fix the sutures automatically without tying any knot.
